(19) United States
(12) Patent Application Publication
(10) Pub. No.: US 2007/0218509 A1
(43) Pub. Date: Sep. 20, 2007

(54) Murine Living Tissue Model & UsesTthereof

(76) Inventors: Malcolm Briden Hodgins, Central Scotland (GB); David Green-Halgh, Central Scotland (GB); Michael Edward, Central Scotland (GB); Alison Reith, Central Scotland (GB)

Correspondence Address: MORGAN LEWIS & BOCKIUS LLP, 1111 PENNSYLVANIA AVENUE NW, WASHINGTON, DC, 20004, US

(21) Appl. No.: 10/552,607

(22) PCT Filed: Apr. 5, 2004
(86) PCT No.: PCT/GB04/01495

§ 371 Date: Feb. 20, 2007

(30) Foreign Application Priority Data
Apr. 5, 2003 (GB) 0307969.6

(51) Int. Cl. G01N 33/50

(52) U.S. Cl. 435/7.21

(57) ABSTRACT

The invention provides an in vitro method for observing an effect of a test agent on a murine tumour model. The model consists a three-dimensional array of murine fibroblasts in a collagen gel which mimics a connective tissue substrate, on or in which are grown benign or malignant murine tumour cells. The model mimics the interactions between the tumour and the underlying tissue substrate, which in turn influence the effect which potential therapeutic or oncogenic agents have on the tumour tissue. Thus the models of the invention provide more physiologically relevant data than monolayer cultures or other available issue models about the effects a particular test agent will have on tumour tissue in vivo. Preferred embodiments provide a model of epithelial tissue and tumours derived therefrom.

### FIELD OF THE INVENTION

The present invention relates to methods for testing agents for their effect on murine tissue models, and in particular to methods for assessing the effect of test agents on normal tissue or benign or malignant tumour tissue. The model may be used to represent the progression from normal to benign or malignant tumour tissue and is particularly useful for evaluating any therapeutic or oncogenic properties of a given test agent. The invention also relates to murine living tissue models for use in methods of the invention.

### BACKGROUND TO THE INVENTION

Conventional animal tests employed to evaluate new therapeutic anti-cancer agents or identify suspect carcinogens are expensive, time consuming, require skilled animal-trained staff and utilise large numbers of animals. To date in vitro alternatives have relied on the use of conventional cell culture systems which are limited in that they do not allow the three-dimensional interactions that occur between the tumour cells and with their surrounding stromal tissue. This is a serious disadvantage as such interactions are well documented as having a significant influence on the growth and invasion profiles of tumours.

EP0358506 relates to a three-dimensional cell and tissue culture system, based upon a synthetic mesh support on which cells are grown, which may be used in cytotoxicity testing of drugs. There is not however any suggestion of how to test oncogenic properties of a drug and/or potential usefulness of drugs as anti-cancer agents.

### SUMMARY OF THE INVENTION

The present invention provides an in vitro method suitable to allow evaluation of test compounds for oncogenic or anti-cancer properties that can, in part, replace the need to test in live animals.

The invention is based on a model developed using a combination of murine tumour cells (particularly epithelial tumour cells) and stromal cells within a three-dimensional collagen gel that mimics a connective tissue matrix. Thus, the models incorporate the influence of activated stromal cells on the growth and invasion characteristics of specific tumour cells, particularly cell lines, following treatment with novel drugs or exposure to carcinogens in a similar fashion to tumours in vivo. The models may also be used to examine the effects of particular delivery vehicles for therapeutic agents on tumour growth and progression, for example, to compare the effects of the same agent administered via different delivery systems, or simply to assess whether a delivery vehicle itself (e.g. a viral vector) is capable of affecting tumour growth or progression.

The approach described herein comprises a new reproducible method, capable of incorporating computer analysis, of cell growth and invasion. This may be used to highlight the flexibility of this in vitro tumour progression model in the assessment of the role, as an example, of retinoids, well-established therapeutics, and additional genotoxic carcinogens or intrinsic mutations.

In a first aspect, the present invention provides an in vitro method for observing an effect a test agent has on a mouse living tumour model, comprising the steps of:

a) providing at least one three-dimensional mouse living tissue model, wherein said model is intended to model benign and/or malignant tumour tissue, and optionally also normal tissue;

b) contacting the test agent with said model(s); and

c) observing the effect the test agent has on said model(s).

The tissue model is a synthetic construct which comprises a three-dimensional array of fibroblasts in a collagen matrix and at least one test cell. The method comprises observing the effect of the test agent on at least one type of tumour tissue; thus the test cell may be a model of either benign or malignant tumour tissue. However the method may further comprise the step of observing the effect of the test agent on test cells which are a model of normal tissue, e.g. as a control. Furthermore, normal test cells may be included with tumour test cells in order to mimic the interactions between tumour tissue and normal tissue.

The method may further comprise the steps of constructing a living tissue model by contacting a collagen solution with a population of fibroblasts, and allowing the collagen to set into a gel. This results in the formation of a three-dimensional array of fibroblasts in a contracted collagen gel, without the use of non-physiological supports or substrates such as nylon mesh, as used e.g. in the constructs described in EP 358 506 A.

The invention further provides a murine tumour model comprising a three dimensional array of murine fibroblasts in a collagen gel and at least one murine test cell, wherein the test cell is a model of normal tissue or benign or malignant tumour tissue, as described herein.

The test cell or cells may be supported on a surface of the array; in preferred embodiments a plurality of test cells form a layer supported on a surface of the array. Additionally or alternatively the test cell or cells may be located within the array. For example, a number of test cells may be dispersed within the array.

The test cell may be a primary cell or a cell line, although cell lines are preferred in order to minimise the number of animals which must be sacrificed in order to prepare the model.

Preferably, the test cell is an epithelial cell. A suitable model of normal epithelial tissue may be used to form an epithelial layer supported on the surface of the fibroblast/collagen matrix. A model of a benign epithelial tumour will tend to form clumps growing at the surface of the collagen, while a malignant epithelial cell model will tend to invade the collagen substrate.

The model may comprise more than one type of test cell. For example, it may comprise both normal and tumour cells. Additionally or alternatively it may comprise more than one type of tumour cell.

Thus, in the case of an epithelial tumour model, the test cells may comprise both normal epithelial cells and epithelial tumour cells, in order to mimic the interactions between normal epithelial cells and the tumour cells as well as those between the stromal cells and the tumour cells.

It is well known that tumours are frequently heterogeneous, comprising more than one type of tumour cell at different stages of the tumourigenic process. This may be modelled by providing more than one type of test tumour cell. In a model of a skin tumour, for example, both the SP-1 and T52 hufos cells may be used together as models of benign and malignant cells respectively.

The test cells and/or the stromal cells may be labelled to allow identification of the test cells. Where the model comprises more than one type of test cell, each type may be labelled with different labelling agents to facilitate separate identification of each type.

The model may be used to study the effects of a given test agent on a test cell of any desired tissue type. In preferred embodiments, the fibroblasts and test cells are derived from the same tissue type, as described in more detail below.

The method may comprise the additional step of selecting an agent which has a desired effect on the test cell.

Mouse cells are used in this model because of the importance of the mouse in studies of carcinogenesis. The model can be constructed of cells from specific genetically modified mice, thereby making use of this important, expanding animal resource, while minimising the numbers of animals needed. Alternatively cells from normal, genetically unmodified mice may be used. As a further alternative, cells may be used which have been genetically modified in vitro. Typically the fibroblasts are primary cells, and are preferably embryonic or neonatal fibroblasts. In preferred embodiments these are combined with normal, benign and/or malignant mouse epithelial cell lines to create epithelial tissue models.

The present in vitro model is intended in part to replace or reduce existing tests carried out on live mice via a pre-screening service. It is likely that some testing on living mice will still need to be conducted to validate results as a companion test, but the intention is that this will be reduced.

The test agent may be any agent including chemical agents (such as toxins), pharmaceuticals, peptides, proteins (such as antibodies, cytokines, enzymes, etc.), and nucleic acids, including gene medicines and introduced genes, which may encode therapeutic agents such as proteins, antisense agents (i.e. nucleic acids comprising a sequence complementary to a target RNA expressed in a target cell type, such as RNAi or siRNA), ribozymes, etc. Additionally or alternatively, the test agent may be a physical agent such as radiation (e.g. ionising radiation, UV-light or heat); these can be tested alone or in combination with chemical and other agents. The models described herein may be used to test for an agent's anti-cancer properties or alternatively for any carcinogenic properties of the test agent.

The model may also be used to test delivery vehicles. These may be of any form, from conventional pharmaceutical formulations, to gene delivery vehicles. For example, the model may be used to compare the effects on a tumour of the same agent administered by two or more different delivery systems (e.g. a depot formulation and a controlled release formulation). It may also be used to investigate whether a particular vehicle-could have effects of itself on the tumour tissue or on normal tissue. As the use of gene-based therapeutics increases, the safety issues associated with the various possible delivery systems become increasingly important. Thus the models of the present invention may be used to investigate the properties of delivery systems for nucleic acid therapeutics, such as naked DNA or RNA, viral vectors (e.g. retroviral or adenoviral vectors), liposomes, etc. Thus the test agent may be a delivery vehicle of any appropriate type with or without any associated therapeutic agent.

The “normal”, “benign tumour” and “malignant tumour” models developed by the present inventors are particularly useful in testing a test agent's properties. The models can be used to test for the ability of an agent to promote the conversion of a cell from a normal to a tumour phenotype (either to a benign or a malignant phenotype), or from one tumour phenotype to another (e.g. from a benign to a malignant phenotype), or for a therapeutic effect, such as the inhibition of proliferation, cytotoxicity, or induction of apoptosis in tumour cells.

The mouse living tissue model may be a modified form of established systems which have been used for constructing human dermal equivalents. However, the living tumour tissue model is developed from and comprises mouse cells rather than human cells, as this is a more equivalent replacement to the living mouse carcinogenesis models currently used.

In preferred embodiments, the present invention is directed to the development of mouse epithelial models responsible for all carcinomas, as these are the most common types of tumour. Typically this may include models of skin, other stratified squamous epithelia, mammary, intestinal (e.g. colon) or lung epithelial tissue or tumours. However other types of tumour may also be modelled by the methods of the present invention, including, but not limited to, sarcomas, melanomas or lymphomas. For example, the systems described may be used to model an interaction between an epithelium and a tumour of non-epithelial origin, or originating from a different epithelial type, e.g. a metastasis from a tumour located elsewhere in the body.

A preferred model for use in the present invention comprises a disc, plug or the like of a collagen gel, which may be formed, for example, formed from a solution of collagen into which fibroblasts are mixed. Once it has set, the fibroblasts contract the gel into a connective tissue-like disc. Thereafter the contracted collagen gel or sponge is inoculated/seeded with test cells (e.g. epithelial tumour or normal cells) which adhere to the surface of the collagen or invade into the gel, forming tumour-like clusters or an epithelium characteristic of the tissue of origin. In another embodiment, fibroblasts and test cells (such as tumour cells) are incorporated into the gel from the start, before it sets and contracts. The test cells and optionally the fibroblasts are derived from the appropriate tissue on which the model is to be based. That is, for example, if the model is a skin tumour model, the epithelial cells and optionally the fibroblasts are obtained from a source of skin tissue. Further incubation in culture medium, either submerged or semi-submerged at body temperature for up to three weeks, allows the epithelial cells to grow and establish structures representative of the tumour of origin or normal tissue in vivo.

The collagen is preferably Type I collagen, Type III collagen, or a combination of the two. The collagen solution from which the gel is formed preferably has a collagen concentration of between 0.3 mg/ml and 3.0 mg/ml collagen.

The seeding density of the fibroblasts can be varied, but will typically be in the range of 1×10⁶ to 1×10⁷ cells per ml collagen gel cast.

This protocol has the advantage of providing a matrix which mimics that occurring in vivo, without the use of non-physiological substrates or supports such as nylon mesh, used in other tissue modelling constructs. Such non-physiological substrates typically cannot be degraded by the tumour cells in the same way as a physiological connective tissue matrix. In the present invention, though, the stromal cells are incorporated directly into a contracted gel formed from collagen, which is the major natural component of tissue matrix, and provides a much more physiologically relevant model of the interactions between tumour cells and the underlying tissue.

Further components found in physiological connective tissue may be added to the collagen gel as desired. These ray include molecular components such as hyaluronic acid and chondroitin sulphate, as well as other cellular components such as endothelial cells or lymphocytes, to model angiogenic effects of tumour cells or the reciprocal effects of tumour and immune cells on one another.

In an embodiment of the present invention, the present inventors have developed mouse skin tumour models in which newborn/embryonic mouse skin fibroblasts have been used to produce a contracted collagen gel. This collagen gel has then been utilised to produce three distinct models—“normal”, “benign” and “malignant” epidermal cell tumours.

The mouse epithelial cell line BalbMK may be used to produce, for example, a normal “control” in vitro model. The mouse skin epidermal papilloma cell line SP-1, which carries a mutant c-ras^{Ha} gene, may be used for example, to produce a benign or papilloma model when incorporated into the model. The TS2 Hufos cell line is a variant of SP-1, formed from SP-1 cells which had been transfected with human fos and may be used to produce, for example, a model that is representative of an invasive malignant stage of tumour development. It will be appreciated however, that other suitable cell lines which develop “normal”, “benign” or “malignant” models may be utilised. These may be originally developed from, for example, experimentally induced tumours in epidermis of mice carrying specific genetic alterations, for example, activated oncogenes or deleted tumour suppressor genes. Alternatively, primary tissue may be used, although this is less preferred because of the need for more animals to be sacrificed to construct the model.

It is to be understood that a “normal” model is intended to be equivalent to the tissue architecture from which the cells are taken. As described above, cells intended to represent “normal” tissue may be primary cells or cell lines. Cell lines which can model normal tissue may be capable of indefinite propagation in the laboratory but typically retain fundamental characteristics of normal cells such as contact inhibition (i.e. inhibition of movement and division caused by contact with neighbouring cells), and are not able to form tumours when injected into animals.

Models of benign tumour tissue typically form tumours which have a non-invasive phenotype and do not infiltrate into the collagen substrate. By contrast, models of malignant tissue are invasive and do infiltrate the substrate.

A model of skin tissue may be generated using skin keratinocytes on a collagen gel impregnated with fibroblasts, which leads to a model with a histopathology of a stratified epithelium on a normal dermis. Initially keratinocytes attach to the matrix and once raised to the air/liquid interface they form a basement membrane and begin to differentiate forming a normal epidermis (illustrated in FIG. 2). A “benign” model is intended to be equivalent to tissue in which a benign tumour, for example a papilloma, has developed. For example, the epithelial cells may clump together and grow together at the surface of the collagen, forming papilloma-like structures (see FIG. 3). Finally, a “malignant” tumour model is a model in which the epithelial cells display an invasive nature and infiltrate the collagen gel, such that the epithelial cells do not just remain exposed at the surface of the collagen gel (see FIGS. 1 and 4).

Thus in accordance with the method of the present invention it is preferable that the test agent is tested on at least two of the models described herein, e.g. the “normal” and “malignant” models, or all three types of model, i.e. “normal”, “benign” and “malignant”. In this manner it is possible to determine a test agent's effect on different stages of tumour progression. Typically the different tissue types will be found in separate constructs, e.g. in separate cultures. However it will be apparent that separate constructs need not be used for each model, as it is possible to include two or all three types of cell or tissue in a single model.

The test agent may be added to said model to be tested by any suitable means. For example, the test agent may be added drop-wise onto the surface of the model and allowed to diffuse into or otherwise enter the model, or it can be added to the nutrient medium and allowed to diffuse through the collagen gel to the test (e.g. epithelial/tumour) cells. The model is also suitable for testing the effects of physical agents such as ionising radiation, UV-light or heat alone or in combination with chemical agents (for example, in photodynamic therapy). Multiple models may be set up in, for example, multiwell tissue culture plates, to allow testing of many agents and/or different concentrations under different conditions.

Observing the effect the test agent has on said models may include a variety of methods. For example, a particular agent may induce a test cell to enter apoptosis. Detectable changes in the test cell may comprise changes in test cell area, volume, shape, morphology, marker expression (e.g. cell surface marker expression) or other suitable characteristic, such as chromosomal fragmentation. Cell number may also be monitored in order to observe the effects of a test agent on cell proliferation; this may be analysed directly, e.g. by counting the number of a particular cell type present, or indirectly, e.g. by measuring the size of a particular cell mass, such as a tumour. These may be observed directly or indirectly on the intact model utilising, for example, suitable fluorescent cell staining. This can be by pre-labelling of tumour cells with vital dyes or genetically introduced fluorescent markers (for example green fluorescent proteins) for serial analysis of the living model or by fixation and post-labelling with fluorescent substances such as propidium iodide or fluorescently labelled antibodies. Alternatively, models may be processed by normal histological methods, such as immunohistochemistry, using antibodies directed against a suitable cellular target, or in situ hybridisation, to test for expression of a particular mRNA species. Moreover, this may be carried out in an automated/robotic or semi-automated manner, using computer systems and software to image the cells at various time points and detect any change in, for example, cell density, location and/or morphology. Confocal laser scanning microscopy in particular permits three-dimensional analysis of intact models. Thus it is possible to apply directly to the intact, three-dimensional tumour model, quantitative analysis of cell behaviour which are normally only possible for cells in conventional two-dimensional culture. By this means quantitative, serial analysis of cell proliferation, apoptosis, necrosis, migration and matrix invasion, among others, are obtained in a three-dimensional tumour cell model which bridges the gap between conventional two-dimensional cell cultures and live animal models.

Also, by appropriate control of viewing/photographing the model, such as by viewing/photographing several fields at random and thereafter randomly selecting a subset of these, it is possible to minimise any bias which may be introduced by a person analysing the data. It is also possible to observe if the test agent induces or inhibits cellular production of proteins, using suitable techniques known in the art, for example, using immunohistochemisty, immunofluorescence, PCR, microarrays, immunoblotting and zymography.

The invention further provides a synthetic tissue model as described herein comprising a three-dimensional array of fibroblasts in a collagen matrix and at least one test cell. Preferred features of the model are as described above in relation to methods of the invention.

### BRIEF DESCRIPTION OF THE DRAWINGS

FIG. 1 shows a schematic representation of a model and how it may be utilised according to the present invention. The model (panel (a)) comprises a collagen gel (C) contracted by embryonic or newborn organ-specific stromal cells, which support a layer of either benign or malignant mouse epithelial cells (E). Models are submerged in media (M) for the initial stages of culture (panels (a) and (c)) and are thereafter raised to the air-liquid interface (panels (b) and (d)) to promote cell differentiation. Test agents with potential anti-cancer activity can be added into the model system at different stages of tumour development (panels (a) and (b)) either into the media of the submerged (a) and raised cultures (b) or onto the surface of the model in the raised culture (b). Models can also be prepared incorporating both stromal and tumour cells within the gel (panels (c) and (d)).

FIG. 2: BalbMK “control” mouse immortalised epidermal keratinocyte model: Propidium iodide stained whole mounts (x 100 magnification) of (A) 2 day submerged culture and (B) 9 day submerged culture and H & E stained paraffin sections (×100 magnification) of (C) 2 day submerged culture and (D) 10 day culture which has been raised to the air interface for 4 days.

FIG. 3: SP-1 “papilloma” model: Propidium iodide stained whole mounts (×100 magnification) of (A) 2 day submerged culture (B) 8 day submerged culture (C) 9 day culture which has been raised to the air interface for 1 day and H & E stained paraffin sections of (D) a 2 day submerged culture (×100 magnification) and (E) a 10 day culture which has been raised to the air interface for 4 days, (F) reconstructed three-dimensional confocal image of a model showing SP-1 cells covering the gel surface and piling up into papilloma-like structures.

FIG. 4: T52 Hufos “invasive tumour” model: Propidium iodide stained whole mounts (×100 magnification) of (A) 4 day submerged culture (B) 6 day submerged culture (C) 10 day culture that has been raised to the air interface for 4 days and H & E paraffin stained sections (×400 magnification) of (D) 4 day submerged culture (E) 6 day submerged culture and (F) 10 day culture which has been raised to the air interface for 4 days.

FIG. 5: The effect of 10⁻⁷M retinoic acid on the area of cell cover in the SP-1 papilloma model over a 12 day period, assessed by imaging propidium iodide stained whole mounts.

FIG. 6: The effect of retinoic acid concentration on the area of cell cover in the SP-1 papilloma model at day 8 and day 10 of culture.

FIG. 7: The effect of 10⁻⁷M retinoic acid on the area of cell cover in the T52 Hufos invasive tumour model over a 10 day culture period.

FIG. 8: Detection of apoptotic cells. Condensed and fragmented cell nuclei, characteristic of cell death by apoptosis, can be detected on (A) an H & E stained paraffin section of a T52 Hufos model (×400 magnification) and (B) a propidium iodide whole mount of an SP-1 model (×200 magnification).

FIG. 9 shows vital dye stained whole mounts (×100 magnification) (A, C and E) and H & E stained paraffin sections (×400 magnification) (B, D and F) of submerged cultures of CMT93/69 mouse rectum carcinoma (A & B), CMT64/61 mouse lung carcinoma (C & D) and TA3 Hauschka mouse mammary carcinoma (E and F).

FIG. 10: Treatment of models with the anti-tumour drug cisplatin. H & E stained paraffin sections (×400 magnification) of SP-1 “papilloma” model (A, C and E) and T52-Hufos “invasive tumour” model (B, D and F). Shown are (A and B) untreated models (without cisplatin), (C and D) models treated with 50 μM cisplatin and (E and F) models treated with 500 μM cisplatin.

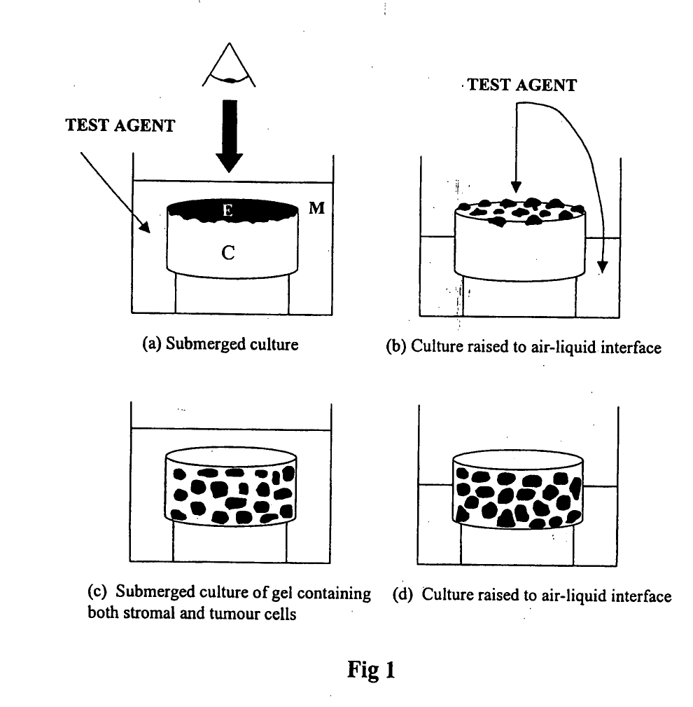

### EXAMPLES

### Example 1

### Preparation of the Living Tissue Model

In summary the in vitro models were developed using contracted collagen gels which supported a layer of mouse tumour cells or corresponding “normal” epithelial cells. The collagen gel was comprised of type I collagen, isolated from rat tail tendon, contracted using primary mouse fibroblasts, which were isolated from the dermis of newborn (or embryonic) mice. The collagen gels contracted to a size approximately 1.5 cm in diameter and were seeded with a single cell suspension of mouse tumour or normal epithelial cells. The models were initially maintained as submerged cultures, which allowed the cells to adhere to the collagen gel and grow. The models may thereafter be raised to the air-liquid interface (semi-submerged culture) to promote cell differentiation and formation of tissue. Benign tumour cells were observed to grow on the surface of the lattice and aggregate to form piles of cells equivalent to wart-like skin papillomas whereas, malignant tumour cells were observed to grow both on top of and into the support matrix mimicking invasive carcinomas. Test agents can be added to the model system at different stages of tumour development. The models are directly imaged as whole mounts by fluorescent labelling of cells either with vital dyes or genetic markers or after fixation and staining.

In more detail, the collagen gel, described below, which supports the layer of epithelial cells (BalbMK (Weissman and Aaronson 1985), SP-1 (Strickland et al. 1988) or T52 Hufos (Greenhalgh and Yuspa 1988) in the prototype models), was contracted using dermal fibroblasts isolated from newborn mouse skin. The epidermis and dermis were separated from each other following an overnight digestion with trypsin at 4° C. The dermal tissue washed in sterile PBS, dissected-into very small pieces, suspended in 3-5 ml of MEM culture media and the slurry seeded into a 75 cm² plastic culture flask. This tissue was cultured without disturbance until the tissue pieces had adhered to the plastic flask. Fresh MEM (Minimum Essential Medium) supplemented with 10% foetal calf serum, 1% L-glutamine, 100 U/ml penicillin and 100 μg/ml streptomycin was added to the flask at this time. Fibroblast cell outgrowth from the dermal tissue explants was observed after several days. The excess tissue was removed and the fibroblast cells cultured to confluence. Collagen gels were prepared by mixing type I collagen solution, with ten-fold concentrated MEM and foetal calf serum containing mouse fibroblast cells in a ratio of 4:1:1. The fibroblast density and collagen I concentration can be varied.

The collagen gels contracted from 3.5 cm diameter to 1.5 cm diameter using the optimised fibroblast seeding density of 0.3×10⁶ fibroblasts per ml of collagen gel cast. Collagen gels were placed into 24 well culture dishes and seeded with epithelial cells. Cell seeding densities were optimised so that the epithelial cells produced measurable areas of cell cover. The seeding density was optimised to 0.5×10⁶ cells/collagen gel. This seeding density ensures that there are sufficient cells present to form a good area of cell cover on the collagen lattice at the time of seeding. This is important, as the cells require cell-cell contact for further growth and differentiation on the collagen lattice support.

Models were routinely cultured for four days as submerged cultures. The models were then raised to the air interface by placing them onto porous stainless steel mesh or sintered glass supports with media reaching to the base of the epithelial cell layer. This allowed the model to continue to receive the nutrients from the culture medium as well as the support and the nutrients generated from the collagen lattice.

Three different mouse skin epithelial cell lines were used to create prototype in vitro models with different properties. The BalbMK cells, which are slow growing mouse immortalised keratinocytes, grow in medium with a low concentration of calcium (0.05 mM) supplemented with 5 ng/ml epidermal growth factor (EGF) (Weissman and Aaronson 1985). BalbMK cells, although immortalised, represent a normalised epithelial cell line and are used in the in vitro model system to provide a “normal”, control model. The mouse skin epidermal papilloma cell line SP-1 carries a mutant c-ras^{Hs} gene. SP-1 cells grow in a low calcium environment and have papilloma-like qualities and are used to provide a benign tumour model (Strickland et al. 1988). The T52 Hufos cell line is a variant of SP-1 cells, which has been transfected with human fos, and grows in the same media as SP-1 cells” supplemented with G418 and are used to provide a malignant tumour model (Greenhalgh and Yuspa 1988). The models may easily be adapted to provide other models comprising alternative mouse epithelial cell lines of, for example, skin, mammary, intestinal or lung origin.

### Example 2

### Processing of Models and Data Interpretation

Cell models were harvested at appropriate time intervals and fixed overnight, at room temperature in a solution of buffered formalin. A small piece of the model was removed and embedded in paraffin wax. Sections were cut and mounted onto glass slides and stained with haematoxylin and eosin (H & E stain). The remainder of the model washed in PBS (phosphate buffered saline), permeabilised with Triton X-100 and stained with propidium iodide (PI). The washed models were stored thereafter in the dark at 4° C. and maintained their fluorescence for several weeks. Whole mounts of the PI stained models were analysed using fluorescent microscopy through coverslips applied directly to the surface of the tissue.

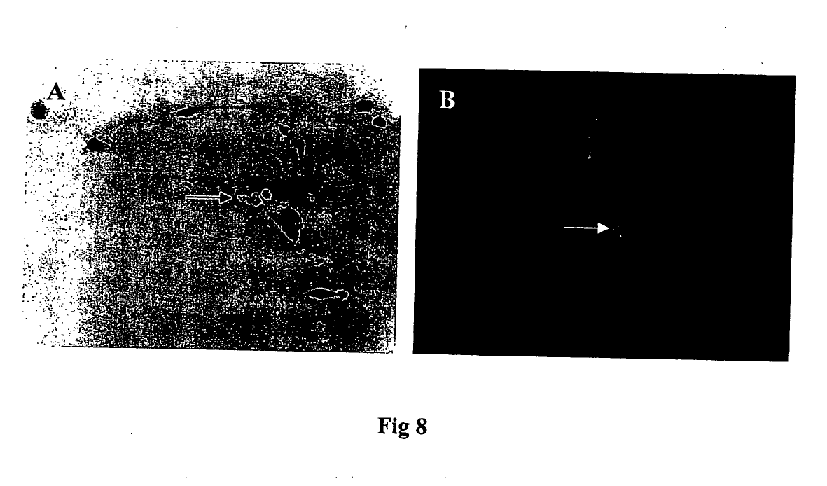

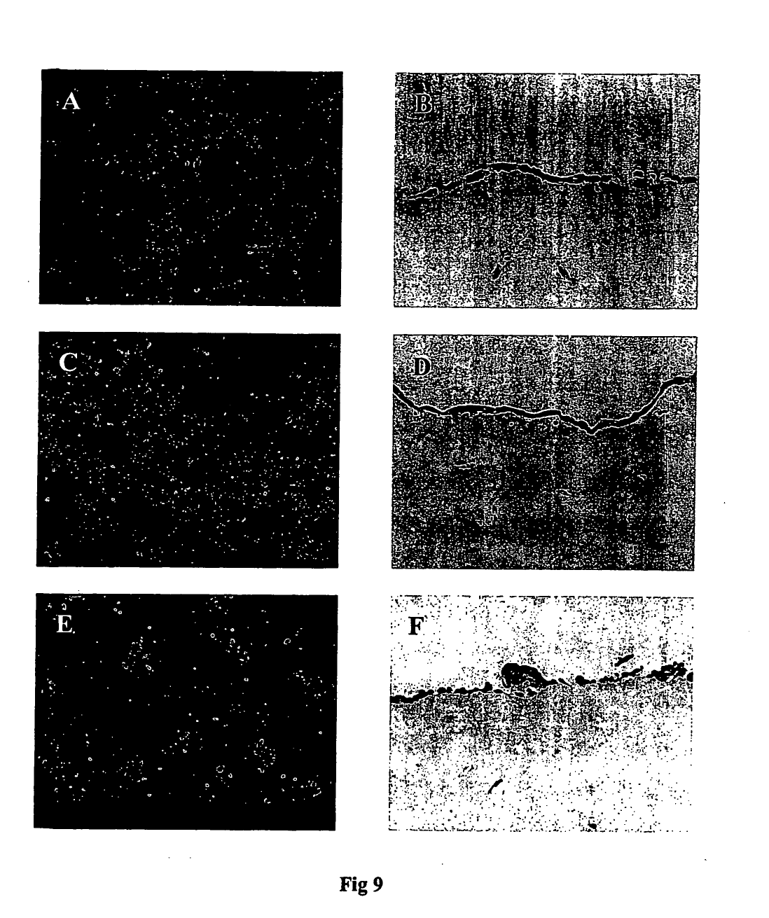

Propidium iodide staining of the whole mounts allows changes in the cell nuclei to be observed. Condensation and fragmentation of the cell nucleus, indicative of cell death by apoptosis can be clearly identified FIG. 8). Haematoxylin/eosin staining identified changes in cell morphology, cell spreading, differentiation and cell death. Alternatively, tumour cells were pre-labelled with the fluorescent dye “DiI” before incorporation into the model. Labelled tumour cells could then be imaged directly in the living model (FIG. 9).

The model is also suitable for applying immunohistological methods of detection to look for specific proteins, which may be altered by a specific test reagent.

Four random fields of view were selected for each model and photographed at ×100 magnification. The areas of cell cover were measured using the computer graphics package Adobe Photoshop. Images were downloaded as picture files directly into this programme. The PI stains all the cell nuclei red and, as only one colour is present, these areas can be selected on the basis of the colour intensity. The percentage area of cell cover is quickly calculated from this data. The data obtained is analysed using Minitab Statistical softwear (Minitab Inc).

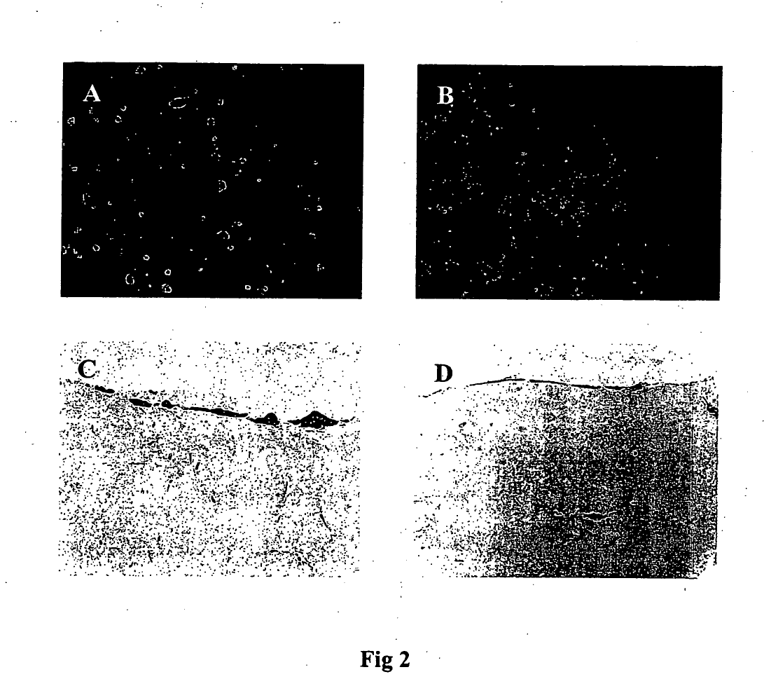

BalbMK cells initially form small clusters on the surface of the collagen gel in a submerged culture (FIG. 2). These clusters become less defined on prolonged culture as the cells spread out and form a more even monolayer (FIG. 2). The morphology of the BalbMK cells in this model resembles a simplified epithelium. This model represents a normal “control” murine living epithelium equivalent.

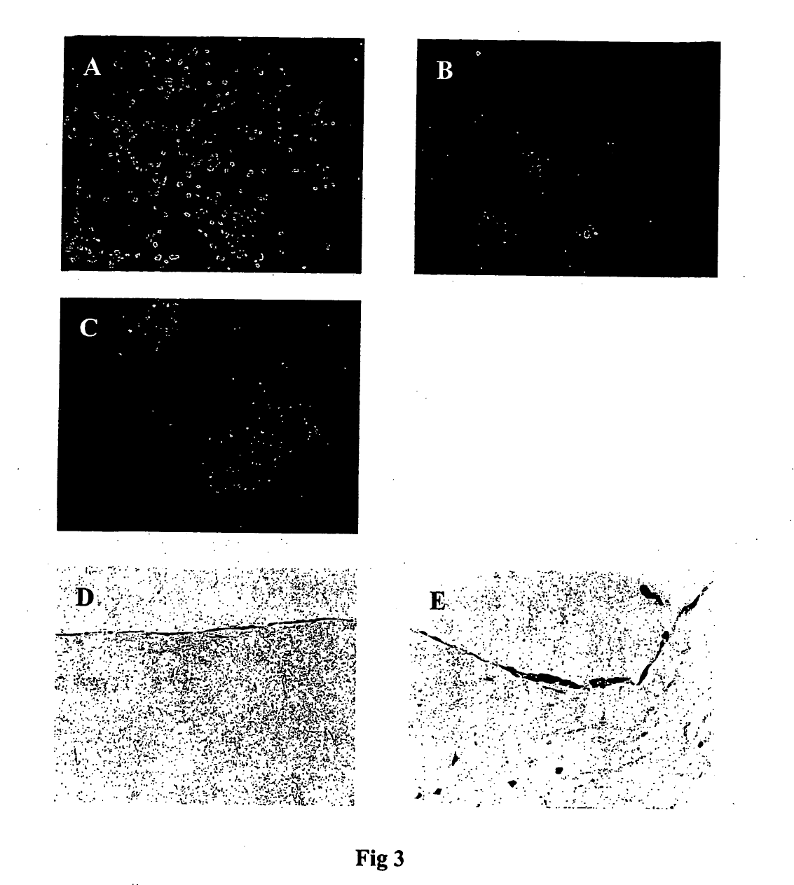

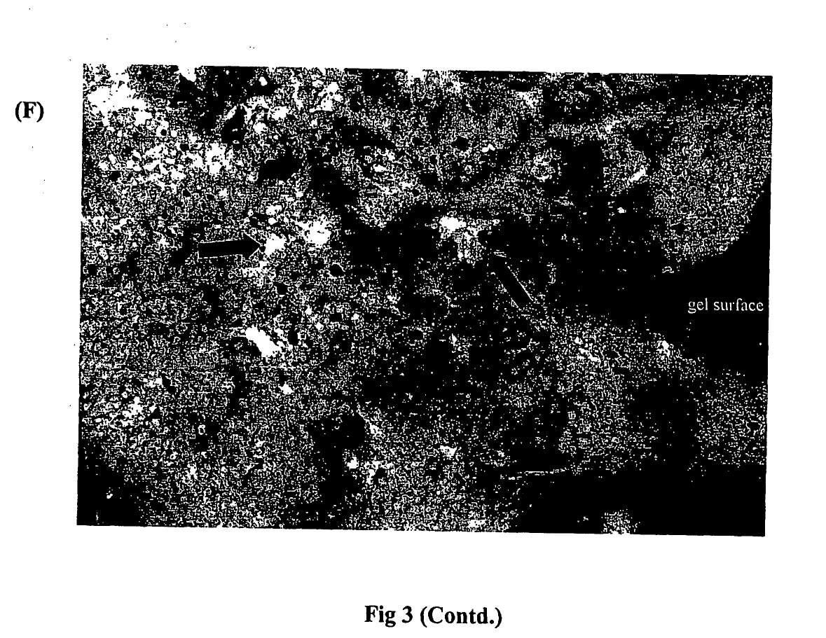

SP-1 cells adhere to the surface of the collagen gel and spread out across the surface (FIG. 3). After 5 days in culture the cells start to retract and by 8 days have formed distinct clusters (FIG. 3). The cells stack to form clusters 3-4 cells thick. Raising the culture to the air interface promoted the formation of these “papilloma” structures. This model shows the benign papilloma stage of tumour progression.

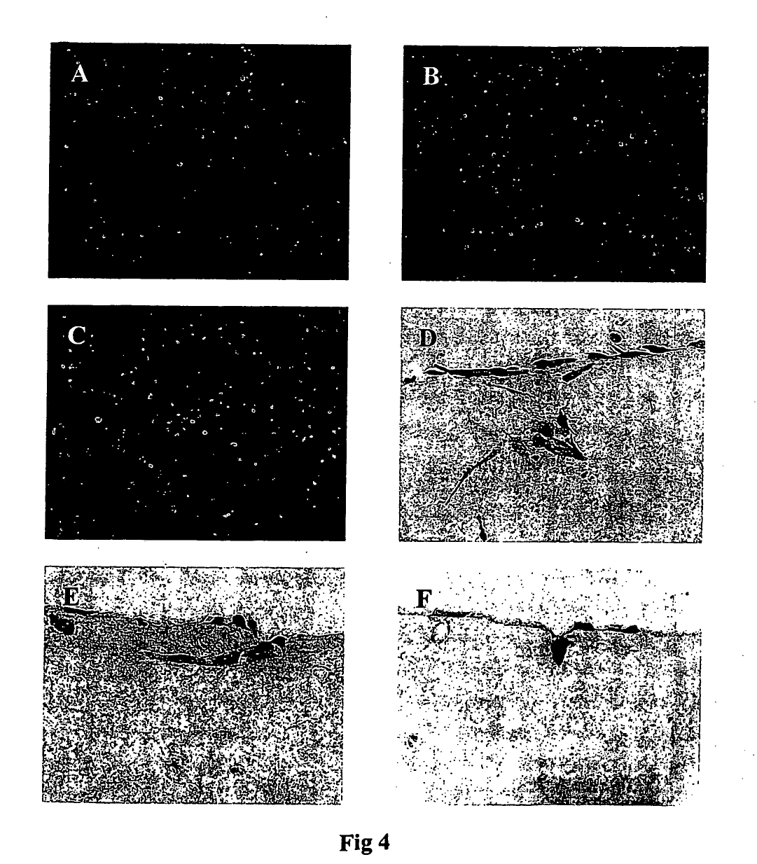

T52 Hufos cells adhere to the surface of the collagen gel and spread out across the surface of a submerged culture (FIG. 4). The cell cover decreases with time in culture (FIG. 4). Viewing this model in cross section shows that the cells have invaded into the collagen gel after 3 days in culture (FIG. 4). This model shows an invasive, malignant phenotype typical of a carcinoma.

### Example 3

### Reproducibility and Viability of the Models

Reproducibility studies were performed using both BalbMK and SP-1 models grown over a time course and harvested as submerged cultures at day 3 and day 4 and raised to the air interface at day 3, 4, 5 and 6. Models were set up in quadruplicate. Four fields of view were photographed for each of the 48 different models and the area of growth measured in each one. Mean areas and standard errors were calculated for each group. Fields of view compared from within the same gel gave similar areas of growth. Measurements made of different models, which were cultured under the same conditions, gave comparable results. There were instances where areas measured were different from their replicates. This was due primarily to the presence of a large cluster of cells. Replicate models are routinely set up for all treatments studied and several fields of view are studied for each model to minimise errors introduced by natural variations and to act as a quality control.

Bias of the Data and Methods of Trying to Overcome This

As the cells on the surface of the models frequently form patterns or interesting morphologies there was a danger that a bias may be introduced when photographing the models to the areas of greatest interest. This has-been overcome by photographing several fields of view at random followed by randomly selecting a subset of these in an attempt to minimise any bias introduced by the person analysing the data. The models often showed unusual cell distributions at the edge of the model. These areas were avoided when measurements were being made. Samples were recorded using number codes to minimise bias in the interpretation of the data.

### Example 4

### Use of the Model to Evaluate Test Agents

All-trans-Retinoic acid (RA) was selected as the first test agent for use in this system. RA is a well characterized agent which has been shown to inhibit mouse skin papilloma growth and has been used extensively in monolayer culture.

To test RA in the model, a single concentration of RA was selected and the models harvested over a time course. SP-1 models were set up and cultured in submerged conditions for 4 days. The cultures were treated with 10⁻⁷M RA and gels were harvested and fixed over a time course at days 6, 7, 10, 11 and 12, corresponding to 2, 3, 4, 5 and 6 days with RA respectively. 10⁻⁷M is the concentration that is routinely used in monolayer cultures but as the three-dimensional in vitro models reflect many properties of in vivo tissue it is possible that higher concentrations may be required to achieve a similar effect.

A concentration gradient of RA was used in submerged cultures of the SP-1 model. The SP-1 cells were cultured on the collagen lattices and after four days of submerged culture RA was added to the cultures at 10⁻⁶M, 10⁻⁷M and 10⁻⁸M with untreated SP-1 cells as the control. Models were harvested in replicate at 8 band 10 days of submerged culture (corresponding to 4 and 6 days with RA respectively) and the data was processed for analysis.

Morphological differences characteristic of apoptosis, such as size and change in nuclei composition were detected in all models using both the H & E and PI staining methods. This allows cell death by apoptosis to be quantified within the model system.

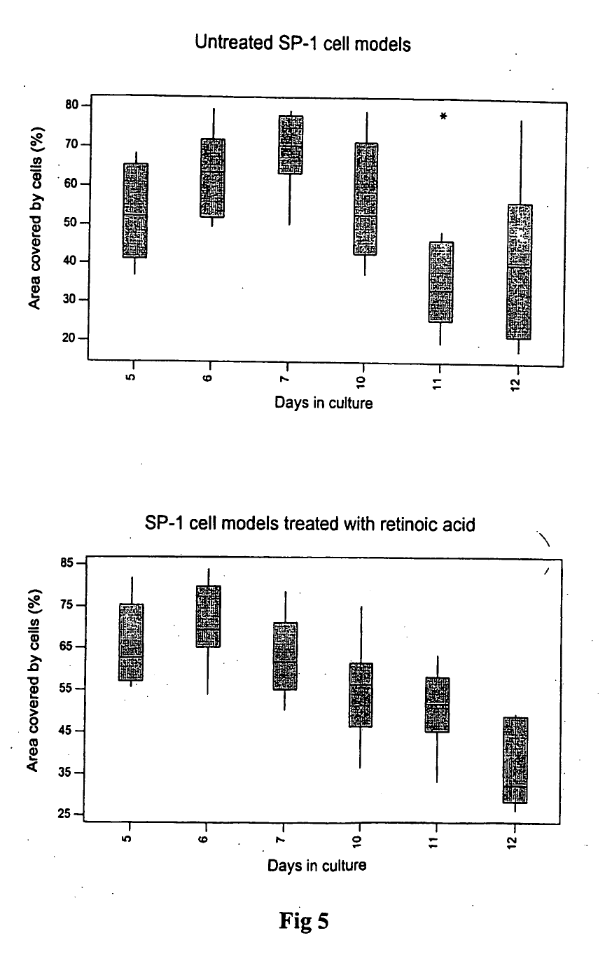

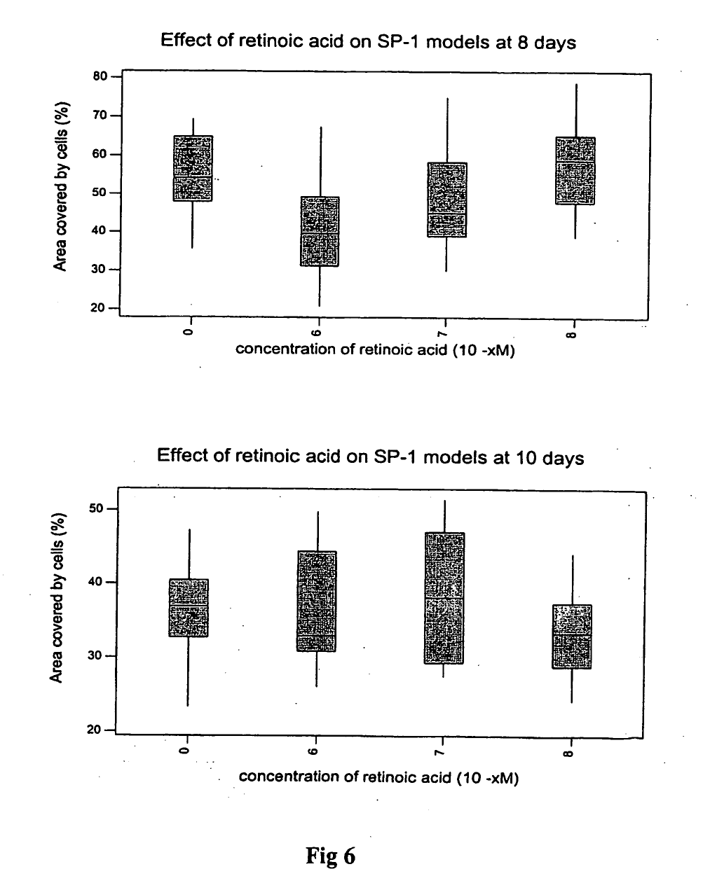

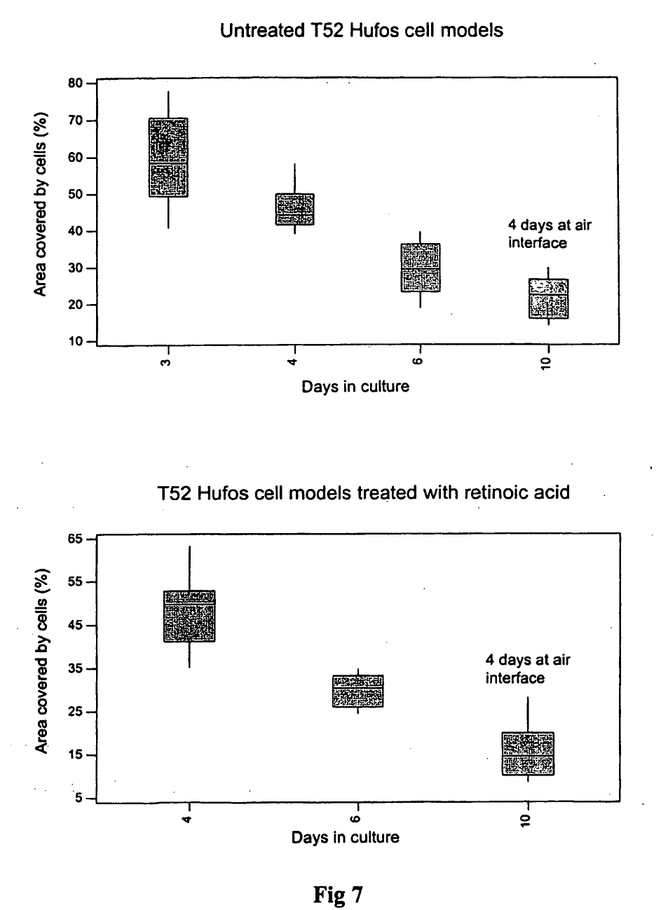

Growth curves of SP-1 cells in monolayer culture showed that retinoic acid had a growth inhibitory effect on SP-1 cells with 46% inhibition of the log phase of growth at 10 days in culture with 10⁻⁷M RA. SP-1 cell models treated with 10⁻⁷M RA showed a modest effect with less cell cover than the corresponding untreated controls (FIG. 5). Studying the effect of a concentration gradient of RA on the SP-1 model showed a marked effect on the model at the highest concentration studied, of 10⁻⁶M RA, with a considerable decrease in cell cover (FIG. 6). The degree of growth inhibition was shown to be concentration dependent. Incorporation of RA into the T52 Hufos model showed no effect (FIG. 7). The T52 Hufos cells showed resistance to RA. These data demonstrate that the SP-1 and T52 Hufos models show different and independent behaviour to the test agent RA.

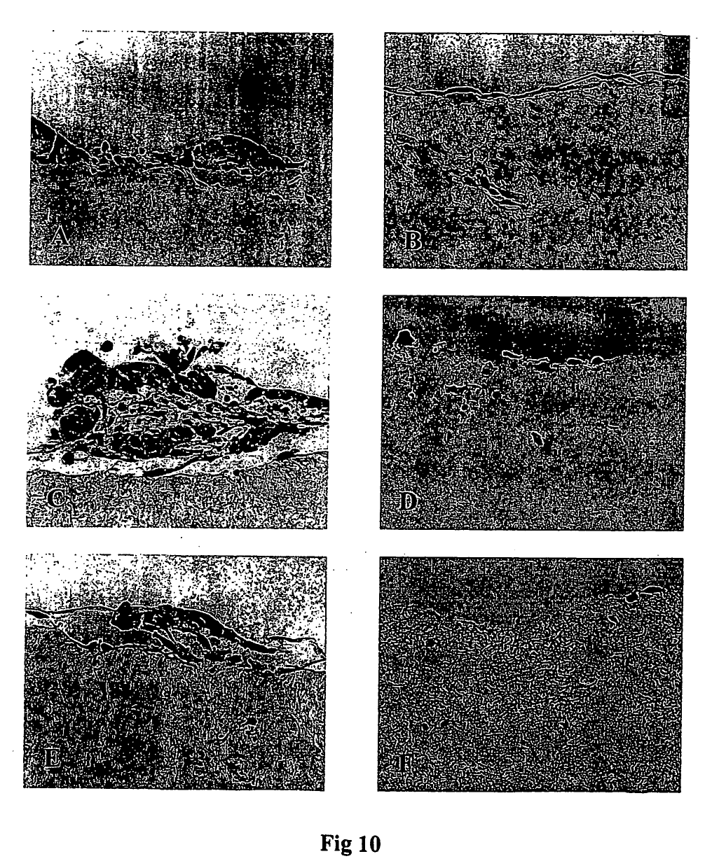

The models provide the ability to readily assess cell killing as well as growth inhibition. cisplatin has been used to demonstrate the effect of a cytotoxic agent in this system. FIG. 10 shows the cell killing effects of two different concentrations of cisplatin on SP-1 papilloma and T52 Hufos invasive tumour models, both of which show evidence of cell killing with the lower cisplatin concentration and extensive cell death with the higher concentration.

### Example 5

### Development of Further Models

Three additional in vitro models have been constructed using mouse tumour cells to diversify the application of this model system for testing anti-cancer therapeutic agents.

All three cell lines tested were of epithelial origin. The CMT 93/69 mouse rectum carcinoma Franks and Hemmings 1977), the CMT 64/61 mouse lung carcinoma (Franks et al. 1976) as described in Example 9 and TA3 Hauschka mouse mammary carcinoma cells (Hauschka 1953; Klein et al. 1972) were seeded onto collagen gels, which had been contracted with embryonic or newborn stromal cells, and cultured as submerged cultures for 6 days. FIG. 9, shows the visualisation of these models both as whole mounts and as H & E paraffin sections. CMT 93/69 rectal carcinoma cells produced an intact epithelial layer-within this system (FIG. 9B). A similar epithelial layer was also observed with the CMT 64/61 lung carcinoma cell line (FIG. 9D). The mouse mammary carcinoma TA3 Hauschka produced an epithelium which was more clustered in appearance (FIGS. 9E and 9F) and some cellular invasion was observed.

Advantages of the System:

The present system provides an effective in vitro replacement for animal testing for new anti-tumour agents in a living mouse model and also provides a good replacement for current monolayer culture assays. The fibroblasts utilised in these models were obtained from the dermis of newborn mice, a single litter providing enough cells for circa 200 gels. The in vitro model has the advantage that it takes considerably less time than an animal model to yield data. The models are quick and easy to set up and require only days to produce the relevant papilloma and carcinoma models, in comparison with animal studies where several weeks are required before SP-1 cells generate a papilloma at the graft site. Shorter culture periods are desirable for productivity and rapid turnover of data.

Moreover, this novel method of analysis and detection allows effects to be observed in three dimensions, vertically and horizontally. This allows total cell coverage on the surface of the model to be quantified (horizontal two-dimensional measurements) as well as the amount of invasion into the support gel. Fluorescent vital cell labelling allows analysis' (vertical three-dimension measurements) of cell dynamics in the living tumour models. This approach lends itself to simultaneous automated dynamic monitors of multiple tissue models for testing purposes.

Test agents can be introduced into the model once the benign (eg. SP-1 model) or carcinoma (eg. T52 Hufos model) structures have formed, and studied for their effect on established growth. Alternatively the test agent can be introduced at the earlier stages of development in the model. This flexibility of the model has the potential to provide valuable data on the mode of action of a specific test agent.

Used in combination, a benign model and malignant model can be used as a conversion assay to study the conversion of cells from the benign papilloma stage of development to the invasive, malignant stage. This has valuable implications for studying new therapeutic agents as it will aid in the determination as to what stage in tumour development a new drug requires to be administered. Ultimately, this system will allow a wide range of new therapeutic agents to be evaluated for their efficiency as anti-cancer agents.

The model system has been designed to screen new compounds that have not been characterised previously with respect to their activity towards carcinogenic tissue. By testing the new agent in the presently described models, it could be provided to the customer with a breakdown of how this new agent will behave against benign “papilloma-like” cell clusters as well as against the later malignant carcinoma stage. The diversity of this system will allow not only the changes in cell cover to be determined but also changes in the cell phenotypes, observations of cell death, differentiation, and invasion (or lack of it). The use of a combination of the benign and malignant models will allow the agent to be tested for its effectiveness during the conversion phase of a tumour from a benign to a malignant phenotype. This is of significant importance as some test agents may work more effectively at the early stages of tumour development rather than at the later stages. The implications of such a model for testing the potential therapeutic properties of a test agent is also valuable.

While the invention has been described in conjunction with the exemplary embodiments described above, many equivalent modifications and variations will be apparent to those skilled in the art when given this disclosure. Accordingly, the exemplary embodiments of the invention set forth are considered to be illustrative and not limiting. Various changes to the described embodiments may be made without departing from the spirit and scope of the invention. All references cited herein are expressly incorporated by reference.

### REFERENCES

Franks L. M., Carbonell A. W., Hemmings V. J., Riddle P. N. (1976). Metastasizing Tumours from Serum-supplemented and Serum-free Cell Lines from a C57BL Mouse Lung Tumor. Cancer Res. 36: 1049-1052
Franks L. M., Hemmings V. J. (1978). A cell line from an induced carcinoma of mouse rectum. J. Pathol. 124: 35-38.
Greenhalgh D. A., Yuspa S. H. (1988). Malignant Conversion of Murine Squamous Papilloma Cell Lines by Transfection with the fos Oncogene. Molecular Carcinogenesis 1: 134-143.
Hauschka T. S. (1953). Cell Population Studies on Mouse Ascites Tumors. Trans. N.Y. Acad. Sci. 16: 64
Klein G., Friberg S., Harris H. (1972). Two Kinds of Antigen Suppression in Tumor Cells Revealed by Cell Fusion. J. Exp. Med. 135: 839-849.
Strickland J. E., Greenhalgh D. A., Koceva-Chyla A., Hennings H., Restrepo C., Balaschak M., Yusap S. H. (1988). Development of Murine Epidermal Cell Lines Which Contain an Activated rash^{Ha} Oncogene and Form Papillomas in Skin Grafts on Athymic Nude Mouse Hosts. Cancer Res 48: 165-169.
Weissman B., Aaronson S. A. (1985). Members of the src and ras Oncogene Families Supplant the Epidermal Growth Factor Requirement of BALB/MK-2 Keratinocytes and Induce Distinct Alterations in Their Terminal Differentiation Programme. Mol. Cell. Biol. 5: 3386-3396.

1. An in vitro method for observing an effect of a test agent on a murine tumour model, comprising the steps of:
a) providing at least one synthetic murine living tissue model comprising a three-dimensional array of murine fibroblasts in a collagen gel and at least one murine test cell, wherein the test cell is a model of benign or malignant tumour tissue;
b) contacting the test agent with said model(s); and
c) observing the effect the test agent has on said test cell.

2. A method according to claim 1 wherein the test cell is supported on a surface of the array.

3. A method according to claim 2 wherein a plurality of test cells form a layer supported on a surface of the array.

4. A method according to claim 1 wherein the test cell is located within the array.

5. A method according to claim 1 wherein the fibroblasts and test cells are derived from the same tissue type.

6. A method according to claim 1 wherein the test cell is an epithelial cell.

7. A method according to claim 6 wherein the test cell is from skin, mammary, lung, or intestinal epithelium.

8. A method according to claim 1 wherein the model comprises more than one type of test cell.

9. A method according to claim 8 wherein the model comprises a normal test cell and a benign and/or malignant tumour test cell.

10. A method according to claim 1 wherein the test cell is labelled.

11. A method according to claim 1 wherein the test agent is a chemical agent, pharmaceutical, peptide, protein or nucleic acid or radiation.

12. A method according to claim 1 wherein the test agent is a delivery vehicle for a therapeutic agent.

13. A method according to claim 1 comprising determining the effect of the test agent on test cell number, area, volume, shape, morphology, marker expression or chromosomal fragmentation.

14. A method according to claim 1 further comprising the step of selecting an agent which has a desired effect on the test cell.
